(19) United States
(12) Patent Application Publication
(10) Pub. No.: US 2012/0277306 A1
(43) Pub. Date: Nov. 1, 2012

(54) COMPOSITION CONTAINING METAL-ACIDIC AMINO ACID CHELATE ACCELERATING ABSORPTION OF METAL

(76) Inventor: Myunggyu PARK, Yongin-Si (KR)

(21) Appl. No.: 13/542,922

(22) Filed: Jul. 6, 2012

Related U.S. Application Data
(62) Division of application No. 12/307,611, filed on Jan. 6, 2009, now abandoned.

(30) Foreign Application Priority Data
Jul. 13, 2006 (KR) 10-2006-0065671

(51) Int. Cl. A61K 31/315; A61P 15/00

(52) U.S. Cl. 514/494

(57) ABSTRACT

Provided is a method for treating prostate and/or testicular disease, comprising: administering a pharmaceutical composition comprising a zinc-aspartate chelate as an active ingredient to a subject in need thereof, wherein the prostate disease is selected from the group consisting of prostatitis, enlargement of prostate and prostate cancer. The composition for accelerating absorption of metals, comprising a metal-acidic amino acid chelate as an active ingredient. The composition of the present invention has therapeutic effects on prevention and treatment of various diseases arising from a deficiency or shortage of metals, by utilization of a metal-acidic amino acid chelate that exhibits excellent effects of improving delivery and absorption of a drug into target organs, such that pharmaceutical effects of individual metals can be exerted. In particular, a pharmaceutical composition comprising a zinc-aspartate chelate increasing an intraprostatic zinc content as an active ingredient is highly effective for prevention and treatment of prostate and testicular diseases.

### CROSS REFERENCE TO RELATED APPLICATION

This application is a Divsional of co-pending U.S. application Ser. No. 12/307,611 filed on Jan. 6, 2009, which is the National Phase of PCT International Application no. PCT/KR2007/003179 filed on Jun. 29, 2007, which claims priority from Korean Patent Application No. 10-2006-0065671 filed on Jul. 13, 2006. The entire contents of all the above applications are hereby incorporated by reference.

### FIELD OF THE INVENTION

The present invention relates to a composition for accelerating absorption of metals, comprising a metal-acidic amino acid chelate as an active ingredient. More specifically, the present invention relates to a composition for accelerating absorption of metals, comprising a complex (chelate) of an acidic amino acid and a metal as an active ingredient, wherein the chelate exhibits an excellent activity by having superior improvements in delivery and absorption of metals into target organs so as to exert pharmaceutical effects of individual metals, and a pharmaceutical, sitological or cosmetic composition comprising the same. Among the compositions of the present invention, particularly the pharmaceutical composition comprising a zinc-aspartate chelate capable of increasing an intraprostatic zinc content as an active ingredient is highly effective for prevention and treatment of prostate and testicular diseases.

### BACKGROUND OF THE INVENTION

The prostate is part of a man's sex organs and makes a viscous fluid. It's about the size and shape of a walnut and surrounds the tube called the urethra, located just below the bladder. The prostate functions to provide seminal fluid in the ejaculate that nourishes and supports the sperm. It is common for the prostate gland to grow and become enlarged as a man ages. In most of mammals, the urethra, the tube through which urine empties from the bladder, passes through the prostate. Due to such anatomical characteristics of the prostate, males often suffer from problems associated with urination as they grow older.

In the male, the prostate is a main organ responsible for pathogenesis of various diseases including prostate diseases such as prostatitis, enlargement of prostate (such as benign prostatic hyperplasia), and the like. The prostate disease may include prostatitis, benign prostatic hyperplasia and prostate cancer. The prostatitis is defined as infection or inflammation of the prostate gland, usually caused by bacteria. Even though high fever, illness from fatigue and stiffness, caused by the prostatitis, may be acute, these symptoms are usually chronic. Accordingly, it can be said that the prostatitis is an incurable disease with a relatively high recurrence, even though it is treated with a standard therapy. According to statistical investigation, over 30% of males at around the age of from 20 to 50 suffer from prostate diseases, and these prostate diseases account for more than 25% of cases in outpatients with urinary diseases. That is, prostate diseases are common diseases with a very low-complete recovery, so about 80 to 90% of the prostate patients suffer from highly severe problems associated with a relapse of the disease.

Pathogenic causes of the prostate diseases, which were elucidated and understood up to now, include bacterial infections, accounting for about 10% of the total cases, and non-bacterial causes without clear identification. Treatment of the prostate diseases is usually carried out by administration of certain antibiotics and anti-inflammatory agents, and by physical treatments.

The prostatitis is caused by inflammation of prostate tissues, and is characterized by manifestation of symptoms such as frequent urination, weakening and thinning of urination flow, painful micturition, unpleasant pain in abdominal and perineal regions, and extremely heavy testicular pain or lumbago. Further, these symptoms become severe after alcoholic drinking or overwork, which may thereby result in systemic symptoms such as sexual dysfunction, premature ejaculation and physical fatigue.

In particular, chronic prostatitis is very common in the adult male sex. The prostatitis is substantially linked to all of prostate cancer, and the tissue examination on the prostatitis lesion may reveal inflammation even when there are no other noticed medical findings such as apparent manifestations and symptoms. The chronic prostatitis is a deep-seated disease having a significant impact on quality of life in prostatitis patients, even though it may not bring about significant and fatal symptoms in the male. Further, it is difficult to make a correct diagnosis and further difficult to achieve a treatment of the chronic prostatitis.

Attack of the chronic prostatitis is commonly accompanied by symptoms such as difficulty in urination, impotence and sterility, starting from pelvic pain. The urinary difficulty, which is a typical condition caused by the prostatitis, exhibits various symptoms such as sleep loss due to frequent night urination, weakening of urination flow, and intermittent urination where the stream is interrupted. Additionally, the prostatitis is also associated with the conditions including impotence, erectile dysfunction and male infertility.

In this connection, even though there are introduced various methods for treatment of prostatitis and related diseases, merely minorities of them are known to exhibit limited effects on treatment of prostatitis. In addition, none of standard therapies and regimens improve health of the prostate.

The prostatic hyperplasia is an illness that frequently attacks most of the male over the age of 50. As men get older, an androgen in the prostate, i.e. testosterone, converts into dihydroxytestosterone (DHT) at a high rate. The conversion of testosterone into DHT occurs primarily because of an increasing concentration of reductase, an enzyme that mediates conversion of testosterone into DHT, as men get older. DHT effectively binds to a prostate cell receptor, which in turn leads to enlargement of prostate. Onset of prostatic hypertrophy including benign prostatic hypertrophy (BPH) is accompanied by various inconveniences and unbearable symptoms, and may also require a surgical operation in severe cases. It is estimated that about 400,000 patients have annually undergone surgical operations for benign prostatic hypertrophy.

As an approach for the treatment of prostatitis, Baert, L. et al reported to achieve a high antibiotic concentration in a prostate fluid and excellent therapeutic effects, by direct injection of gentamicin into the prostate (Urology 21, 370, 1983).

Masanori Yamamoto et al performed direct injection of amikacin and cefazolin into the prostate of 25 patients to investigate effects of such drugs on chronic bacterial prostatitis, thus confirming therapeutic effects in 56% of subject patients (J. Urol. Nephrol., 30, 199-202, 1996). Korean Patent Application No. 1998-0020895 proposes a method of maximizing therapeutic effects of a drug by direct injection of an antibiotic and an anti-inflammatory agent in the form of a sustained-release formulation to the prostate.

As described above, the treatment of prostatitis generally employs antibiotics and anti-inflammatory drugs. In order to help the treatment of prostatitis, psychotherapy for mental stabilization or administration of anti-depression drugs may also be performed simultaneously. The period necessary for the treatment of prostatitis is at least about 6-8 weeks, even though some cases may take a prolonged period of time. A more serious problem is in that the patient cannot be completely cured due to a difficulty to achieve a complete recovery.

A primary method for treating prostatic hypertrophy focuses on facilitation of urination by alleviating tension of the prostate region via drug medication. However, when such a drug treatment is not effective, prostatectomy may be conducted using an endoscope.

Therapeutic drugs for prostatic hypertrophy are largely α-reductase blockers. For example, HYTRIN (terazosin HCl, Abbot Laboratories), CARDURA (doxazosin mesylate, Roerig), FLOMAX (tamsulosin HCl, Boehringer Ingelheim Pharmaceuticals, Inc.) and the like are commercially available on the market. These drugs improve urethral voiding by relieving muscular tension of the prostate. Unfortunately, these drugs sometimes cause adverse side effects and fail to prevent a recurrence of disease concerned. As another drug used to treat prostatic hypertrophy, PROSCAR (finasteride, available from Merck) serves to facilitate excretion of urine by contracting prostate tissues. However, it was not still revealed that such a drug is practically beneficial to treat prostatitis.

Further, Allopurinol is also used as a therapeutic agent for treatment of prostatitis. This drug lowers a concentration of uric acid in vivo to thereby prevent the occurrence of inflammation which may take place due to formation of uric acid in the prostate. Further, as an allopathic therapy, symptoms of prostatitis are mitigated by massaging the prostate region with hot water, even though there is a limitation in fundamental remedy of the disease.

For treatment of prostate cancer, hormonotherapy is adopted to suppress production processes of androgens or block the action of androgens on the prostate, since prostate cancer cells proliferate largely under the influence of androgens. In addition, radiotherapy may also be employed for cases of symptoms where the hormonotherapy is not effective or when therapeutic effects of hormonotherapy diminish or vanish.

As discussed hereinbefore, direct injection of antibiotics into the prostate to treat prostate diseases is advantageous to directly deliver a high concentration of a drug without causing systemic side effects, due to direct injection of the drug into a target lesion, but suffers from a disadvantage associated with a short working time of the drug.

Meanwhile, a representative example of natural components used to treat prostate diseases is zinc. Generally, zinc is accepted to have positive effects on relief of prostatic hypertrophy.

Upon reviewing conventional prior arts relating to whether zinc is practically effective in the treatment of prostate diseases, U.S. Pat. Nos. 5,234,698, 5,071,658 and 4,946,688 have confirmed that zinc exhibits an anti-bacterial activity and anti-proliferative effects on prostate cells, through direct administration of zinc into the prostate of patients with prostatitis or prostatic hypertrophy via an injection route.

Further, Cho et al have confirmed through various experiment the fact that the incidence of inflammation is effectively inhibited in inflammation-induced rats, by examination of anti-inflammatory effects of zinc and changes in an intraprostatic zinc concentration after administration of zinc to the prostate of the animal via an injection route (see International Journal of Antimicrobial Agents, 19, 576-582, 2002).

Leslie et al have demonstrated that a transporter protein responsible for influx of zinc in prostate cells is absorbed by a different transporter protein that is not metallothionein (J. Inorg. Biochem. 98, 2004, 664-666).

Costello et al have confirmed distribution of citric acid and zinc concentrations depending upon prostate tissues and conditions of diseases, thereby revealing the citric acid-zinc relationship according to individual diseases (Prostate Cancer & Prostatic Diseases, 2004, 7, 111-117).

As discussed above, zinc is known to have therapeutic effects on prostate diseases. In spite of such a fact, available therapeutic drugs are limited because the prostate is located in a deep site of the body and prostate cells have a unique envelope that allows poor passage of drugs. Further, absorption of the drug into the prostate is not easy, thereby making it difficult to achieve effective treatment. In particular, treatment of prostatitis may bring about undesirable drug resistance due to chronic administration of the antibiotic, and may also suffer from a problem that the in vivo balance may be collapsed due to a decreased number of normal bacteria.

In other words, oral administration of zinc is not easy due to poor absorption of zinc into the prostate, so there are still no effective methods for treatment of prostate diseases utilizing zinc, except for injection of zinc. However, infusion of zinc via an injection route suffers from limitations in practical application thereof due to various problems such as the risk of infection, a difficulty in injection, and discomfort and pain to patients.

### SUMMARY OF THE INVENTION

Accordingly, it is an object of the present invention to solve the above problems, and other technical problems that have yet to be resolved.

As a result of a variety of extensive and intensive studies and experiments to solve the problems and disadvantages as described above, the inventors of the present invention have confirmed that a metal-acidic amino acid chelate exhibits excellent absorption effects thereof into the target organ and therefore is remarkably effective for prevention and treatment of various diseases arising from a deficiency or shortage of metals or various diseases requiring a supply of metals. The present invention has been completed based on these findings.

That is, the present invention has been made in view of the above problems, and it is an object of the present invention to provide a composition comprising a metal-acidic amino acid chelate having prophylactic and therapeutic effects on various diseases arising from a deficiency or shortage of metals or various diseases requiring a supply of metals, as an active ingredient, and a pharmaceutical, sitological or cosmetic composition comprising the same.

It is another object of the present invention to provide a pharmaceutical composition comprising, as an active ingredient, a metal-acidic amino acid chelate, particularly a zinc-aspartate chelate, in order to achieve normal development of genital organs, functional maintenance of the prostate, production and activity of gonadal hormones (e.g. gonadotropin), production of insulin, synthesis of nucleic acids and proteins, a certain action on the gustatory center and periphery, growth and development of infants, and enhancement of immune functions, or in order to exert therapeutic effects on various diseases and conditions such as underdevelopment of infants, recession of sexual function, anorexia, prostate disorders, thyroid hyperfunction, a decreased resistance to diseases, gustatory dysfunction, glucose metabolic abnormalities, diabetes, alopecia and the like, which may occur due to the deficiency of a certain metal such as zinc.

It is a further object of the present invention to provide a pharmaceutical composition for prevention and treatment of prostate and/or testicular diseases, comprising the aforesaid zinc-aspartate chelate as an active ingredient.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic view showing the action of aspartic acid and zinc in a synthetic process of citric acid;

FIG. 2 is a photograph showing staining results of prostate tissues in a group with administration of a zinc-aspartate chelate, a control group and a normal group;

FIG. 3 is an enlarged photograph (×200) showing staining results of prostate tissues in a group with administration of a zinc-aspartate chelate and a control group;

FIG. 4 is a graph showing a plasma zinc concentration over time, according to the form of administered zinc;

FIG. 5 is a graph showing zinc concentrations in various organs at 8 hours after administration of zinc, according to the form of administered zinc;

FIG. 6 is a graph showing an intraprostatic zinc concentration over time, according to the form of administered zinc;

FIG. 7 is a graph showing an intratesticular zinc concentration over time, according to the form of administered zinc;

FIG. 8 is a photograph showing expression levels of mRNAs for ZnT-2, ZnT-4 and G3PDH, according to the form of administered zinc; and

FIG. 9 is a graph showing changes in weight of prostate cancer cells in a group with administration of a zinc-aspartate chelate and a control group.

### DETAILED DESCRIPTION OF PREFERRED EMBODIMENTS

In accordance with an aspect of the present invention, the above and other objects can be accomplished by the provision of a composition for accelerating absorption of metals, comprising a metal-acidic amino acid chelate as an active ingredient.

The composition for accelerating absorption of metals in accordance with the present invention has excellent effects on prevention and treatment of various diseases arising from a deficiency or shortage of metals, or various diseases requiring a supply of metals.

There is no particular limit to the aforementioned acidic amino acid. Examples of the acidic amino acid may include glutamate, aspartate, and the like. These amino acids may be used alone or in any combination thereof. Particularly preferred is aspartate.

The aforementioned metal may be a metal having a valence of 2 or more, such as calcium, copper, zinc, iron, chromium, cobalt, manganese, magnesium, and the like. Preferably, the metal is any one selected from divalent metals. Particularly preferred is zinc.

There is no particular limit to the method for preparing the metal-acidic amino acid chelate. Therefore, various methods known in the art may be employed to prepare such a chelate. Particularly preferably, the desired chelate may be prepared by the method of Korean Patent Application No. 2003-0088214, assigned to the present applicant, the disclosure of which is hereby incorporated by reference in its entirety.

The compositions of the present invention may further comprise one or more pharmaceutically acceptable carriers. Such a pharmaceutical composition may be formulated into various dosage forms, depending upon desired applications. Therefore, the present invention further provides pharmaceutical compositions and preparations comprising the above-mentioned metal absorption-accelerating composition.

For preparation of the pharmaceutical composition, the metal-acidic amino acid chelate as the active ingredient is mixed with a variety of pharmaceutically acceptable carriers which may be appropriately selected depending upon the desired formulation to be prepared. For example, the pharmaceutical composition in accordance with the present invention may further comprise conventional diluents or vehicles such as filling agents, extenders, binding agents, wetting agents, disintegrating agents, surfactants and the like, and may be formulated into various preparations such as injectable preparations, oral preparations or the like, depending upon the desired applications. Particularly preferred are oral preparations.

Solid formulations for oral administration include, for example, tablets, pills, powders, granules and capsules, and are prepared by mixing the composition or chelate of the present invention with one or more inert diluents such as sucrose, lactose and starch, lubricating agents such as magnesium stearate, and other carriers such as disintegrating agents, binding agents and the like. Further, liquid formulations, such as suspensions, solutions for internal use, emulsions and syrups, may further comprise various excipients, for example wetting agents, sweetening agents, aromatics and preservatives, in addition to diluents such as water, liquid paraffin, and the like.

Formulations for parenteral administration may include sterile aqueous solutions, non-aqueous solvents, suspensions, emulsions, lyophilized formulations and suppositories. As non-aqueous solvents and suspending solvents, there may be used propylene glycol, polyethylene glycol, vegetable oil such as olive oil, injectable ester such as ethyl oleate, etc. As the base material for the suppository formulation, Witepsol, macrogol, Tween 61, cacao butter, laurin butter, glycerol or gelatin may be used.

A suitable dose of the pharmaceutical composition may vary depending upon various factors such as formulation methods, administration modes, age, weight and sex of patients, pathological conditions, diet, administration time, administration routes, excretion rates and sensitivity to response.

Dosage units of the pharmaceutical composition may contain one-, two-, three- or four-fold amount of an individual dose, or ½, ⅓ or ¼-fold amount of an individual dose. Preferably, the individual dose contains an amount of the active drug that is administered one time, and typically corresponds to the total amount of a daily dose, or ½, ⅓ or ¼-fold amount thereof.

For adults, the dose of zinc as the zinc-aspartate chelate may be perferably in the range of more than 15 mg/day/kg bw, more preferably more than 20 mg/day/kg bw and may be administered 1 to 6 times a day.

The metal absorption-promoting composition in accordance with the present invention may further comprise one or more sitologically and/or cosmetically acceptable carriers.

For example, the composition containing the sitologically acceptable carrier may be used as a health care food or otherwise may be added thereto. As used herein, the term “health care food” refers to a food in which the composition of the present invention is added to a general food to thereby improve functions thereof. The metal-acidic amino acid chelate-containing composition may be added to general foods or may be prepared in the form of capsules, powders, suspensions and the like. Intake of such a health care food containing the metal-acidic amino acid chelate provides advantages in that there are no adverse side effects which may occur upon chronic use of drugs because a food is used as the raw material, unlike conventional drugs.

If it is desired to use the metal-acidic amino acid chelate of the present invention as a food additive, the chelate may be added alone, or otherwise may be used in conjunction with other foods or food ingredients, or may be used appropriately according to any conventional method. A mixed amount of the active ingredient may be suitably determined depending upon the purpose of use (prophylactic, health or therapeutic treatment).

Generally, upon production of foods or beverages with which the chelate of the present invention is mixed, the metal-acidic amino acid chelate may be added in an amount of 0.0001 to 10% by weight, preferably 0.1 to 5% by weight, based on the total weight of raw materials. However, when prolonged intake is intended for the purpose of health and hygiene or for health control, the amount of the chelate may be adjusted below the above-specified range. In addition, the pharmaceutical composition preferably contains the metal-acidic amino acid chelate in an amount falling within the determined toxicity range, when the composition of the present invention is employed as the pharmaceutical composition as mentioned above.

There is no particular limit to kinds of the above-mentioned foods. As examples of foods to which the metal-acidic amino acid chelate can be added, mention may be made of meats, sausages, breads, chocolates, candies, snacks, confectionaries, pizzas, Ramen, other noodles, gum, dairy products including ice creams, various soups, beverages, teas, drinks, alcoholic beverages and multi-vitamin preparations. More specifically, mention may be made of health care foods and special favorite foods such as squeezed liquid, tea, jelly and juice made of the metal-acidic amino acid chelate as the main ingredient.

Further, the present invention provides a composition which comprises a zinc-aspartate chelate exhibiting an activity thereof by effectively increasing a content of zinc and is intended for preventing and treating developmental abnormality of genital organs, abnormal production or activity of gonadal hormones, abnormal production of insulin, weakening of immune functions, recession of sexual function, anorexia, prostate disorders, thyroid hyperfunction, a decreased resistance to diseases, gustatory dysfunction, glucose metabolic abnormalities, alopecia and the like.

According to the facts which were elucidated hitherto, zinc is known to have various pharmacological actions in vivo, such as normal development of genital organs, functional maintenance of the prostate, production and activity of gonadal hormones (e.g. gonadotropin), production of insulin, synthesis of nucleic acids and proteins, a certain action on the gustatory center and periphery, growth and development of infants, enhancement of immune functions, and the like. Therefore, deficiency of zinc may cause diseases and conditions such as underdevelopment of infants, recession of sexual function, anorexia, prostate disorders, thyroid hyperfunction, a decreased resistance to diseases, gustatory dysfunction, glucose metabolic abnormalities, diabetes, alopecia and the like.

As the zinc-aspartate chelate in accordance with the present invention exhibits excellent improvements in delivery and absorption of zinc into the target organ, the aforementioned composition comprising such a chelate achieves effective in vivo delivery of zinc to thereby exert various pharmacological actions of zinc as mentioned above, and therefore is highly effective for prevention and treatment of the above-mentioned diseases and disorders which may be caused by a shortage or deficiency of zinc.

In another preferred embodiment, the present invention provides a pharmaceutical composition for prevention and treatment of prostate and/or testicular diseases, comprising the zinc-aspartate chelate as an active ingredient.

According to the experiments performed by the present inventors, it was confirmed that the aforesaid zinc-aspartate chelate effectively delivers zinc to thereby exert remarkable effects on prevention and treatment of prostate and/or testicular diseases. The prostate disease may be prostatitis, enlargement of prostate (such as benign prostatic hyperplasia) or prostate cancer.

In this connection, one of the most important biological functions of the prostate is synthesis and secretion of citrate. As shown in Reaction 1 below, the citrate synthesis takes place using aspartate as a substrate.

Glucose+2 Aspartate+2O₂→2 Citrate+2CO₂+b 14 ATP (1)

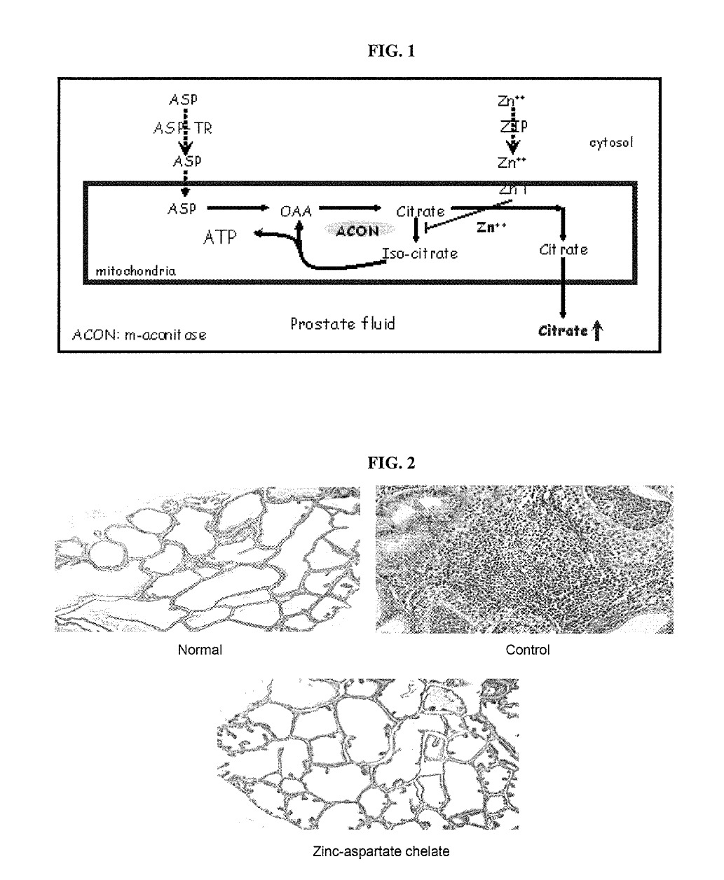

Referring to FIG. 1 showing the action of aspartate and zinc in the synthetic process of citrate, the synthesis of citrate in the prostate requires that aspartate is present at a high concentration, and aspartate in prostate cells is an essential amino acid and therefore must be supplied from blood, unlike other tissue cells. It was recently found that a high-affinity L-aspartate transporter, EAAT3, is present in the prostate (Costello L C et al. BMC Biochemistry 2006, 7:10).

With the study of Zaichick et al (Int. Urol. Nephrol. 1997, 29, 565-74), it was analyzed that a zinc concentration was maintained at a very high level in the prostate, as compared to other tissues or organs, and dry weight zinc concentrations in normal, BPH and cancerous prostate were 1018±124, 1142±77, and 146±10 μg/g, respectively.

From analysis of zinc and cadmium concentrations in tissues of patients with benign prostatic hypertrophy (BPH) or enlarged prostate and prostate cancer by Brys et al (Biol. Trace Elem. Res. 1997, 59, 145-152), it was found that BPH patients exhibit an increase in the zinc concentration, whereas prostate cancer patients exhibit a decrease in the zinc concentration, and both groups exhibit a high concentration of cadmium. Through these results, they reported that cadmium interferes with the function of zinc in the prostate to thereby play an important role in variations of the prostate.

Fahim M S, et al (Andrologia, 1993, 25, 369-375) have reported that zinc has been implicated in steroid endocrinology of the prostate gland. That is, 5 alpha-dihydrotestosterone (DHT) is believed to express androgenic responses in the prostate. From experiments to examine the effects of zinc in rats, the results indicated significant reduction of 5-alpha-reductase activity, prostate weight, total protein and DNA concentrations in treated prostate tissue; and no significant changes in histological structure of testes, epididymides, and seminal vesicles. These findings are the direct results showing effects of zinc on the prostate. That is, these results suggest that direct application of zinc to the prostate can offer a new modality for treatment of prostate diseases including prostatitis without affecting spermatogenesis.

According to the study of Pei Feng (41st Annual Meeting, 2001), the normal prostate gland accumulates 3-10 times more zinc than any other tissue in the body. In contrast, the zinc level in prostate cancer cells is very low. Malignant prostate cancer cells have lost the ability to accumulate zinc in the prostate. The treatment of prostate cancer cells with a high concentration of zinc inhibits cell growth to thereby result in apoptosis or programmed cell death. Therefore, these results confirm that zinc apparently maintains the balance between cell proliferation versus cell death in prostate tissue by acting against the mitochondria to thereby increase a release of cytochrome C, which consequently leads to apoptosis in the malignant and hyperplasia.

However, despite such study results, it was found that intake of zinc as an inorganic mineral results in substantially no translocation of zinc into the prostate tissues and therefore it is impossible to achieve therapeutic effects of zinc on prostate and testicular diseases. That is, smooth translocation of zinc into the prostate tissues is very important for treatment of the prostate and testicular diseases.

Therefore, the present invention provides a pharmaceutical composition for prevention and treatment of prostate and testicular diseases, comprising a zinc-aspartate chelate that exhibits an activity thereof by effectively increasing a content of zinc. Preferably, the prostate disease includes prostatitis, enlargement of prostate (benign prostatic hyperplasia) and prostate cancer.

The aforesaid zinc-aspartate chelate has an excellent delivery function thereof into the target organ and can thus effectively deliver zinc ions into the prostate. In addition, it is possible to very effectively prevent or treat testicular diseases and prostate disease such as prostatitis (including bacterial and non-bacterial prostatitis), benign prostatic hyperplasia and prostate cancer, by improving uptake of zinc to thereby effectively increase an intracellular zinc level.

In order to ascertain such a fact, the present inventors conducted various experiments as follows. Inflammation was caused by inoculating E. coli to prostate tissues of experimental animals and animals were fed with physiological saline (Control group) or orally administered with the zinc-aspartate chelate (Experimental group). Thereafter, the prostate tissues were recovered and stained. As a result, it was confirmed that administration of the zinc-aspartate chelate exhibits excellent anti-inflammatory effects.

Further, the following experiments were carried out on human subjects. One group was administered with an antibiotic and an anti-inflammatory drug (Control group), whereas the other group was administered with the zinc-aspartate chelate (Experimental group). Based on the NIH Chronic Prostatitis Symptom Index (NIH-CPSI), statistical analysis was made by the questionnairing of the subjects on 9 items focusing on the three domains of: 1) pain and discomfort; 2) urinary symptoms; and 3) impact on quality of life. As a result, it was confirmed that administration of the zinc-aspartate chelate leads to very excellent effects comparable to those of the control group.

Accordingly, the pharmaceutical composition for prevention and treatment of prostate and testicular diseases comprising the zinc-aspartate chelate as the active ingredient can prevent and treat the prostate and testicular diseases by increasing an intraprostatic zinc concentration, and thus it is expected that such a zinc-aspartate chelate-containing composition can be developed as various therapeutic agents for a variety of diseases associated with the prostate and testicular diseases.

As discussed hereinbefore, the pharmaceutical composition for prevention and treatment of prostate and testicular diseases in accordance with the present invention may also be formulated into various dosage forms other than an injection. Hence, it is possible to solve various problems associated with the risk of infection, a difficulty in injection, and discomfort and pain to patients, which may occur due to the limitation and disadvantage that conventional drug compositions could be administered only by an injection route.

### EXAMPLES

Now, the present invention will be described in more detail with reference to the following Examples. These examples are provided only for illustrating the present invention and should not be construed as limiting the scope and spirit of the present invention.

### Example 1

### Effects of Zinc-Aspartate Chelate in White Rat Model of Chronic Bacterial Prostatitis

### 1. Materials

As experimental animals, 12 to 16-week old Sprague-Dawley male rats, weighing 250 to 300 g, (Daehan Biolink Co., Ltd., Chungchongbuk-do, Korea) were employed for the present experiment after acclimation to a basal diet for one week. These rats were randomly assigned into a normal control group, a physiological saline-administered group and a zinc-aspartate chelate-administered group.

### 2. Chronic Bacterial Prostatitis Model and Administration of Zinc-Aspartate Chelate

A chronic bacterial prostatitis model was established using a white rat, according to a method of Nickel et al (Br. J. Urol. 1990, 66, 47-54). Escherichia coli ATCC 25922, known as a pathogen of prostatitis, was injected into the prostatic urethra at a density of 1×10⁸ cells/mL.

15 mg/kg/day of a zinc-aspartate chelate dissolved in physiological saline was orally administered to prostatitis-induced rats at a given time point. As a control group, zinc-aspartate chelate-free physiological saline was administered to animals. Administration was continued for 30 days. After 30 days, animals of each group were dissected to remove the prostate from which the anterior lobe and the posterior lateral lobe were then isolated. Thereafter, the prostate membrane was completely removed to obtain the prostate only. Samples of the anterior lobe and the posterior lateral lobe from each group were fixed in 10% neutral formalin, embedded in paraffin and stained with hematoxylin and eosin (H & E), followed by microscopic observation of the paraffin-embedded sections.

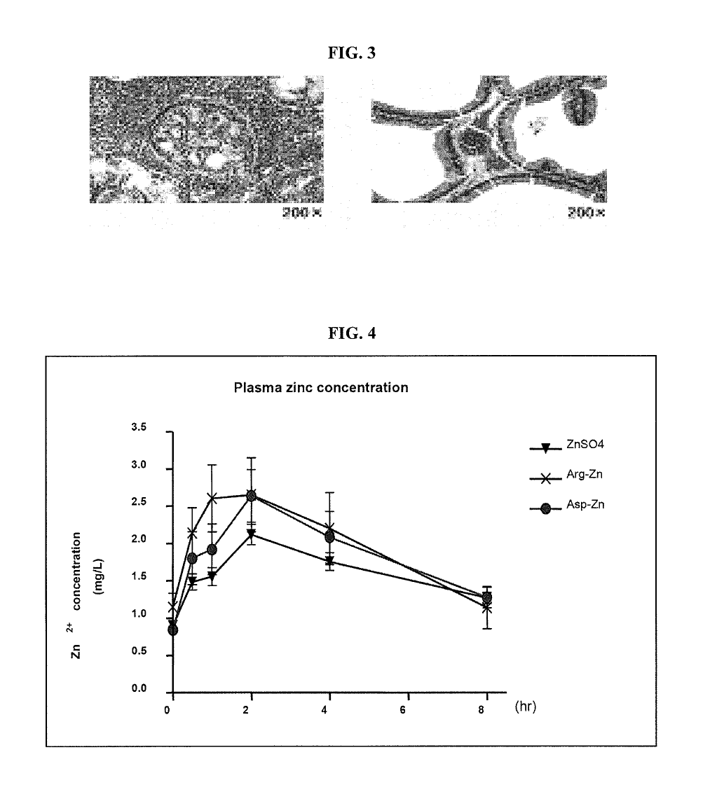

As shown in FIGS. 2 and 3, the infected prostate of the control group exhibited significant infiltration of lymphocytes into the acini and interstitium. On the other hand, the group with administration of the zinc-aspartate chelate exhibited no infiltration of inflammatory cells into and around glandular tissues, exhibited a recovery in the size of the acini and exhibited no lymphocytes and substantially no progress of fibrosis in the interstitium. As a result, the group with administration of the zinc-aspartate chelate exhibited normal tissue morphology comparable to that of the normal group.

### Example 2

### Selective Translocation of Zinc-Aspartate Chelate into Prostate and Testicular Tissues

As experimental animals, 12 to 16-week old Sprague-Dawley male rats, weighing 250 to 300 g, (Daehan Biolink Co., Ltd., Chungchongbuk-do, Korea) were employed for the present experiment after acclimation to a basal diet for one week. These experimental animals were randomly assigned into a zinc sulfate-administered group, a zinc arginine-administered group and a zinc-aspartate chelate-administered group. After animals were fasted for 12 hours, each zinc sample was administered to rats, such that a zinc concentration in the sample is 40 mg/kg. After administration of the samples was complete, blood was collected at a time point of 0.5, 1, 2, 4, and 8 hours, respectively, and treated with heparin. Then, blood plasma was stored in a refrigerator and used for analysis of zinc concentrations. On the other hand, in order to examine distribution of zinc in the tissue, each zinc sample was administered to rats. After 8 hours, animals were sacrificed to remove organs. Zinc concentrations in the blood plasma and tissues were determined by fluorometry using Zinquin.

As shown in FIG. 4, a time-dependent increase of the plasma zinc concentration was confirmed in all of zinc sulfate, zinc arginine and zinc aspartate. In addition, it was confirmed that the zinc arginine- and zinc aspartate-administered groups exhibited a significant increase in the plasma zinc concentration, as compared to zinc sulfate-administered group. Further, the zinc arginine-administered group exhibited a maximum blood zinc level at a time point of 1 to 2 hours after administration of the zinc sample, whereas the zinc sulfate- and zinc aspartate-administered groups exhibited a maximum blood zinc level at a time point of 2 hours after administration of the drug.

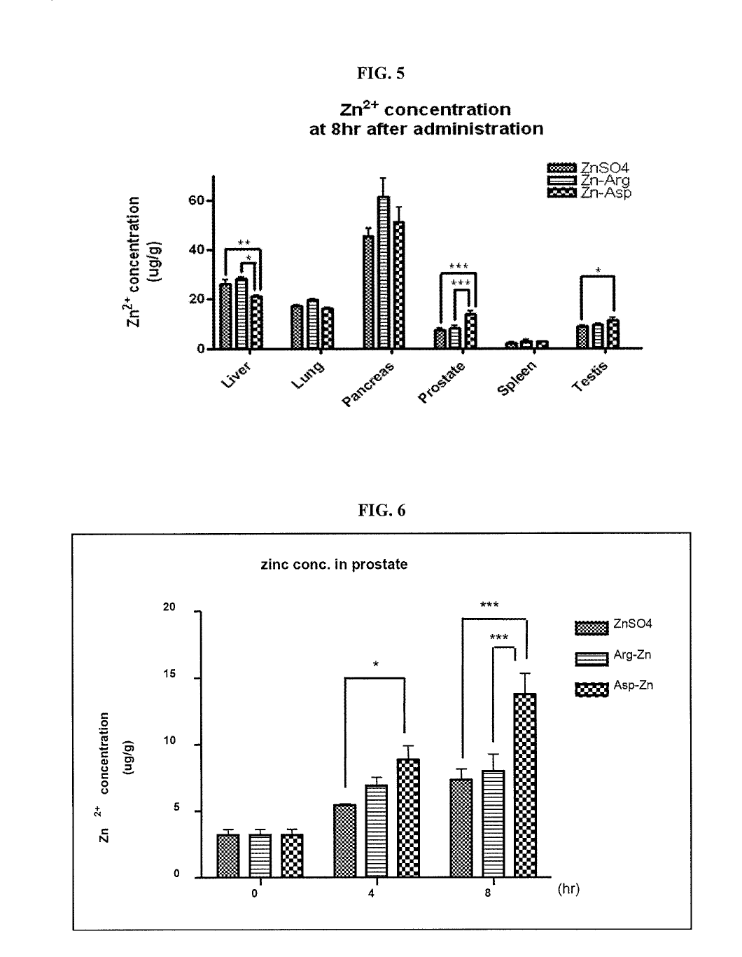

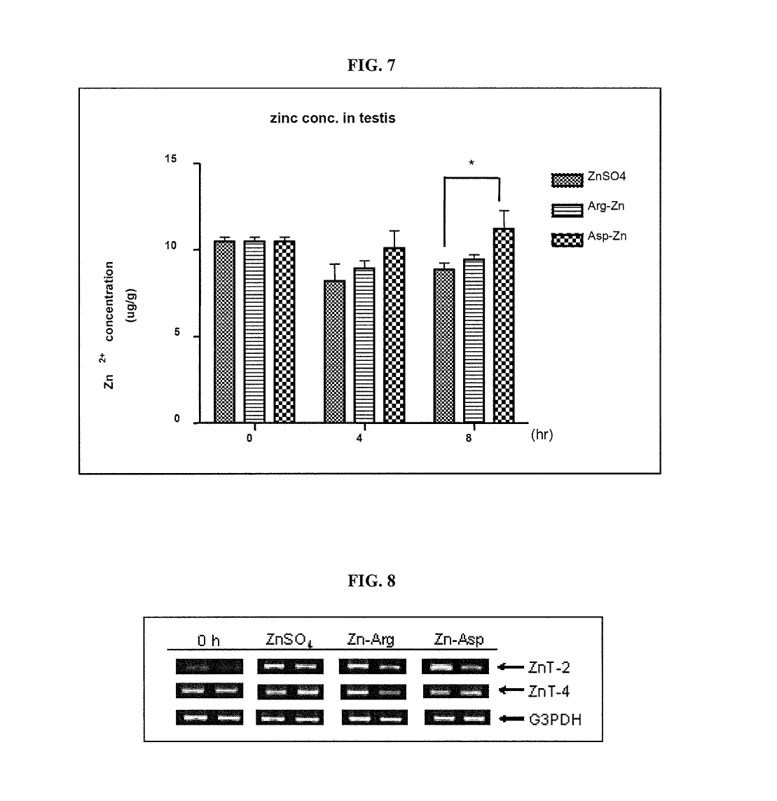

Meanwhile, as shown in FIG. 5, when distributions of zinc in various tissues were compared 8 hours after each sample was administered, most of tissues exhibited similar profiles of zinc distribution therebetween. However, the zinc aspartate-administered group exhibited a significantly high zinc concentration in the prostatic tissue and testis (p<0.001). Further, FIGS. 6 and 7 are graphs showing intraprostatic and intratesticular zinc levels 4 and 8 hours after administration of each sample, respectively. Referring to these drawings, it can be seen that the zinc aspartate-administered group exhibited a significantly high zinc concentration in both the prostatic tissue and testis, as compared to the zinc sulfate- and zinc arginine-administered groups. Based on these results, it can be confirmed that zinc ions are selectively absorbed into the prostate and testis tissues by means of the zinc-aspartate chelate.

### Example 3

### Effects of Zinc-Aspartate Complex on Expression of Zinc Transporter Protein mRNA

In order to examine effects of zinc-aspartate complex on the expression of ZnT-2 and ZnT-4, known as zinc transporter proteins, experiments were carried out according to the following procedure.

Using Trizol Reagent (Invitrogen, USA), total RNA was extracted from 0.1 g of prostatic tissue. Using M-MLV reverse transcriptase (Invitrogen, USA), cDNA was synthesized from 5 μg of the extracted total RNA. 20 pmol of a primer was added to 3 μl of cDNA to make a volume of 20 μl and PCR was carried out as follows: initial denaturation at 95° C. for 5 min, followed by 26 cycles of denaturation at 94° C. for 1 min, annealing at an annealing temperature corresponding to conditions of each primer for 1 min and extension at 72° C. for 1 min, and final extension at 72° C. for 10 min.

Primer sequences and annealing temperatures used in PCR are set forth in Table 1 below.

TABLE 1

| | | | |
|---|---|---|---|
| ZnT-2 | F: | 5′-TCCATCAGTCTGGACATGGA-3′ | 57° C. |
| | R: | 5′-CCCTTCCATGAGAACCAAGA-3′ | |
| ZnT-4 | F: | 5′-ACCTTGCAGCAGGACCTAGA-3′ | |
| | R: | 5′-ATACTGCCGTGGAGAAATGC-3′ | 55° C. |

PCR products were subjected to electrophoresis on 1.5% agarose gel containing EtBr, followed by confirmation of products under UV irradiation.

Effects of the zinc-aspartate complex, zinc sulfate and zinc arginine on the expression of mRNAs for ZnT-2, ZnT-4 and G3PDH were investigated. The results thus obtained are shown in FIG. 8.

Referring to FIG. 8, upon comparing to before oral administration of zinc (Oh), it can be seen that all the groups with oral administration of zinc exhibit an increase in an expression level of ZnT-2, particularly the zinc aspartate-administered group exhibits the highest increase in the expression level of ZnT-2.

### Example 4

### Effects of Zinc-Aspartate Chelate on Prostate Cancer

10-week-old COP male rats (Japan SLC, Shizuoka, Japan) were housed and allowed to acclimate to the new environment for seven days prior to the experiment, in a breeding room maintained at a temperature of 22±2° C., humidity of 55±5%, and a 12-h light/dark (L/D) cycle (light from 8:00 a.m. to 8:00 p.m.).

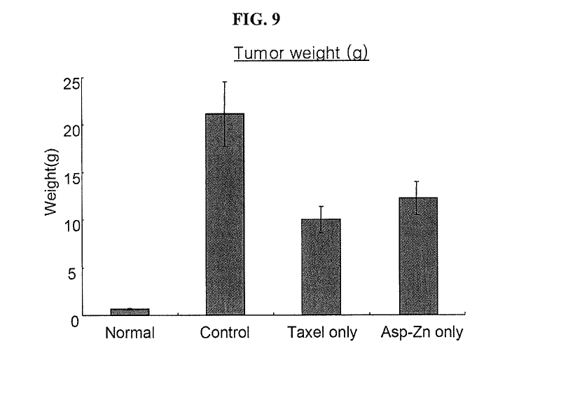

5×10⁴ cells/head of the prostate cancer cell line (R3327 AT-3.1) were injected into the prostate of animals and growth of cancer cells was measured. The results thus obtained are shown in FIG. 9. Drugs and administration regimen are set forth in Table 2 below.

TABLE 2

| Group | Dose (mg/kg) | Remarks (Administration route/Times) |
|---|---|---|
| Control | — | Non-treated |
| DoceTaxel | 10 mg/kg | Once a week, I.P |
| Asp-Zn | 10 mg/kg | Oral. daily |

Referring to FIG. 9, it can be seen that the group with administration of the zinc-aspartate chelate in accordance with the present invention exhibited a significant weight reduction of tumor mass, as compared to the control group, and also exhibited a much lower weight of tumor mass, as compared to the group with administration of commercially available prostate cancer therapeutic drug, e.g. Taxel. Therefore, it can be confirmed that the zinc aspartate in accordance with the present invention can be utilized to treat prostate cancer.

### Example 5

### Clinical Trials of Zinc Aspartate Chelate

34 patients with a medical history of more than 5 years of chronic prostatitis were administered with a zinc-aspartate chelate at a dose of 50 mg/day for 8 weeks. Clinical trials were conducted at the Department of Urology, College of Medicine, Korea University (Seoul, Korea). Prostatitis classification and symptom relief were analyzed according to National Institutes of Health Chronic Prostatitis Symptom Index (NIH-CPSI, proposed by NIH in 1995). As clinical effects by a conventional therapy, the data of the clinical study of Sungkyunkwan University School of Medicine (Seoul, Korea), published in the Korean Journal of Urology (2001), were cited. There were no reports showing noticeable side effects among patients who were administered daily with 50 mg of the zinc-aspartate chelate, and significant therapeutic effects were tested.

Tables 3 and 4 below respectively show the results for chronic prostatitis patients with administration of an antibiotic and an anti-inflammatory drug as conventional therapeutic agents and with administration of the zinc-aspartate chelate, in conjunction with the results for the control group.

TABLE 3
Conventional antibiotic/anti-inflammatory drug for treatment of
chronic prostatitis (therapeutic effects for 4 weeks)

| | n = 14 (male, 38.7 (7.5 years) | | |
|---|---|---|---|
| NIH-CPSI classification | Before treatment | After treatment | P value |
| Pain | 9.18(3.45 | 5.04(2.25 | <0.0001 |
| Dysuria | 5.04(1.62 | 3.39(1.88 | 0.004 |
| Quality of life | 6.39(2.35 | 4.82(1.44 | <0.0001 |

TABLE 4
Zinc-aspartate chelate (8 weeks, oral administration)

| | n = 34 (male, 40.2 (5.3 years)) | | |
|---|---|---|---|
| NIH-CPSI classification | Before treatment | After treatment | P value |
| Pain | 8.412(3.95 | 6.82(4.2 | 0.036 |
| Dysuria | 5.53(2.57 | 4.02(2.87 | 0.01 |
| Quality of life | 3.53(1.39 | 2.61(1.45 | 0.04 |

As shown in Tables 3 and 4, even when comparing with combined effects of conventional drug therapy and physical therapy, administration of the zinc-aspartate chelate in accordance with the present invention provided the results showing almost the similar level of therapeutic effects. Accordingly, it was confirmed that the composition containing the zinc-aspartate chelate is an effective oral composition for treatment of chronic prostatitis which reduces adverse side effects due to abuse of antibiotics or alleviates discomfort and inconvenience of patients and is capable of being used in combination with conventional therapies.

### INDUSTRIAL APPLICABILITY

As apparent from the above description, a metal absorption-promoting composition in accordance with the present invention has therapeutic effects on prevention and treatment of various diseases arising from a deficiency or shortage of metals, by utilization of a metal-acidic amino acid chelate that exhibits excellent effects of improving delivery and absorption of a drug (e.g. a certain metal) into target organs, such that pharmaceutical effects of individual metals can be exerted. In particular, a pharmaceutical composition comprising a zinc-aspartate chelate increasing an intraprostatic zinc content as an active ingredient is highly effective for prevention and treatment of prostate and testicular diseases.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method for treating prostate and/or testicular disease, comprising:
administering a pharmaceutical composition comprising a zinc-aspartate chelate as an active ingredient to a subject in need thereof,
wherein the prostate disease is selected from the group consisting of prostatitis, enlargement of prostate and prostate cancer.

2. The method of claim 1, wherein the pharmaceutical composition is in the form of a pharmaceutical formulation.

3. The method of claim 1, wherein the administration is oral administration.

4. The method of claim 1, wherein the administration is parenteral administration.

5. The method of claim 1, wherein the pharmaceutical composition is designed to administer zinc in an amount of more than 15 mg/day.

6. The method of claim 1, wherein the pharmaceutical composition is targeted into the prostate or gonad.

7. The method of claim 3, wherein the oral administration is selected from the group consisting of tablet, pill, powder, granule and capsule.

8. The method of claim 4, wherein the parenteral administration is selected from the group consisting of sterile aqueous solution, non-aqueous solvent, suspension, emulsion, lyophilized formulation and suppository.
